(19) United States
(12) Patent Application Publication
(10) Pub. No.: US 2010/0280053 A1
(43) Pub. Date: Nov. 4, 2010

(54) CyclopropylmethYl-[7-(5,7-dimethyl-benzo[1,2,5]thiodiazol-4-yl)-2,5,6-trimethyl-7H-pyrrolo[2,3-d]pyrimidin-4-yl]-4-propyl-amine as a CRF Antagonist

(75) Inventors: Philippe SCHÖFFTER, Village Neuf (FR); Regina VOEGELI-LANGE, Basel (CH); Jürgen DRESSEL, Lorrach (DE)

Correspondence Address: Fanelli Strain & Haag PLLC, 1455 Pennsylvania Ave., N.W., suite 400, Washington, DC, 20004, US

(73) Assignee: NOVARTIS AG

(21) Appl. No.: 12/822,697

(22) Filed: Jun. 24, 2010

Related U.S. Application Data
(63) Continuation of application No. 10/544,693, filed on Jul. 26, 2006.

(30) Foreign Application Priority Data
Feb. 7, 2003 (GB) 0302876.8

(51) Int. Cl. A61K 31/519; C07D 487/04; A61P 29/00; A61P 1/00; A61P 25/28; A61P 25/00; A61P 15/00; A61P 3/04; A61P 31/18

(52) U.S. Cl. 514/265.1; 544/280

(57) ABSTRACT

The present invention relates to a novel benzothiadiazole of formula (A) in free base or acid addition salt form, its preparation, its use as pharmaceutical and pharmaceutical compositions containing the compound.

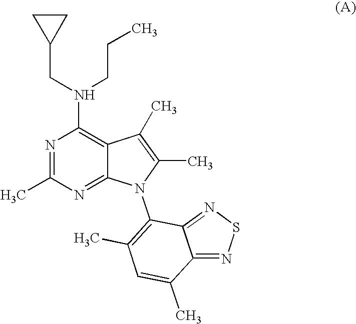

The present invention relates to novel benzothiadiazoles, their preparation, their use as pharmaceuticals and pharmaceutical compositions containing them.

More particularly the invention provides the compound of formula A

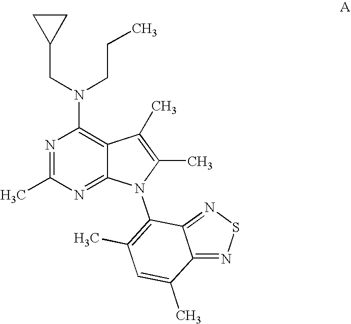

in free base or acid addition salt form.

The compound of formula A is generically embraced by formula I of EP 1 049 694 and equivalent patents or patent applications. This patent family also discloses a process for the production of the compounds of formula I and their acid addition salts, as well as the use of the compounds of formula I in free base or pharmaceutically acceptable acid addition salt form, as pharmaceuticals for the treatment of any state with increased endogenous level of corticotropin releasing factor (CRF) or in which the hypothalamic pituitary'adrenal (HPA) axis is disregulated, or if a disease is induced or facilitated by CRF.

The compound of formula A and its acid addition salts have never been specifically disclosed.

The compound of formula A and its acid addition salts can be prepared by a process including the step of reacting a compound of formula II

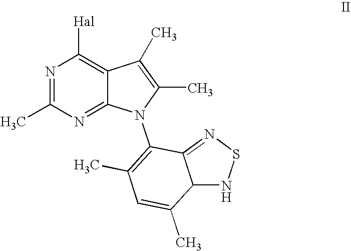

wherein Hal is halogen, with the compound of formula III

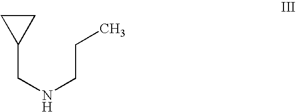

and recovering the resulting compound in free base form or in acid addition salt form.

The reaction may be effected in known manner, e.g. as described in Example 1. Hal is preferably chlorine, bromine or iodine, particularly chlorine.

Working up of the reaction mixture obtained according to the above process and purification of the compound thus obtained may be carried out in accordance to known procedures.

Acid addition salts may be produced in known manner from the free base forms and vice-versa. Suitable pharmaceutically acceptable acid addition salts for use in accordance with the present invention include for example the hydrochloride, the hydrogen maleate, the hydrogen fumarate and the hydrogen malonate.

The starting materials of formula II may be obtained as described in Example 1.

It has now surprisingly been found that the compound of formula A in free base or pharmaceutically acceptable acid addition salt form (hereinafter the agents of the invention) behaves as a non-competitive CRF₁ receptor antagonist.

The non-competitive CRF₁ receptor antagonistic activity of the agents of the invention has been determined in vitro in the following assay:

Chinese hamster ovary (CHO) cells expressing human recombinant CRF₁ (Chen et al., Proc Natl Acad Sci USA 90, 8967-8971, 1993) are propagated in Dulbecco's modified Eagle medium supplemented with 10% foetal calf serum, non-essential aminoacids, 100 U/ml penicillin, 100 mg/l streptomycin and 1 g/l geneticin (G418). For cyblic AMP determinations, cells are grown to confluence in 24-multiwell plates. Stimulation of cyclic AMP accumulation by CRF (human/rat form) is measured in intact cells, using the [³H]adenine labelling technique, as described previously (Schoeffter et al., Neuropharmacology 36, 429-437, 1997).

Concentration-response curves for CRF are constructed in the presence of putative antagonists (1 nM-1 μM) or vehicle (dimethyl sulfoxide 1% vol). IC₅₀ values of antagonists are calculated by fitting the percent inhibition of the effect of CRF (10 nM) by increasing concentrations of the antagonists. The fit is done using the nonlinear logistic function of the Origin software package (OriginLab Corporation, Northampton, Mass., USA).

In this test, the agents of the invention show non-competitive CRF, antagonistic activity with IC₅₀ CRF₁ values of about 1 to 500 nM.

The agents according to the invention are therefore useful in the treatment of any state with increased endogenous level of CRF or in which the HPA (hypothalamic pituitary axis) is disregulated, or of various diseases induced or facilitated by CRF, including inflammatory disorders, such as arthritis, asthma and allergies; anxiety including generalized anxiety; phobic and panic attacks; depression; fatigue syndrome; headache; pain, e.g. inflammatory or neuropathic pain; cancer; irritable bowel syndrome, including Crohn's disease, spastic colon and irritable colon; immune dysfunction; human immunodeficiency virus (HIV) infections; neurodegenerative diseases such as senile dementia, Alzheimer's disease and Parkinson's disease; stroke and head trauma; epilepsy; gastrointestinal diseases; eating and body weight disorders such as obesity and anorexia nervosa; hemorrhagic stress; drug and alcohol withdrawal symptoms; drug addiction; sleeping disorders; hormonal disregulations; skin disorders; stress-induced psychotic episodes; fertility problems; sexual dysfunctions and pre-term birth.

The utility of the agents of the invention in the above indicated diseases could be confirmed in a range of standard tests:

For example the anxiolytic activity of the agents of the invention can be confirmed in the mouse elevated plus-maze [see for example Rodgers R. J., Behavioural Pharmacology 8: 477-496 (1997) where the relevance of the elevated plus-maze is discussed on p. 486; for the method, see Rodgers R. J. et al. Ethology and Psychopharmacology (Eds S J Cooper and C A Hendrie), pp 9-44 (1994), J. Wiley, Chichester]. In this test, the agents of the invention show anxiolytic-like effects on administration of 0.1 to 30 mg/kg p.o.

For the above-mentioned indications, the appropriate dosage will of course vary depending upon, for example, the compound employed, the host, the mode of administration and the nature and severity of the condition being treated. However, in general, satisfactory results in animals are indicated to be obtained at a daily dosage of from about 0.1 to about 100, preferably from about 0.5 to about 100 mg/kg animal body weight. In larger mammals, for example humans, an indicated daily dosage is in the range from about 1 to about 500, preferably from about 1 to about 300 mg of an agent of the invention, conveniently administered, for example, in divided doses up to four times a day or in sustained release form.

The agents of the invention may be administered by any conventional route, in particular enterally, preferably orally, for example in the form of tablets or capsules, or parenterally, for example in the form of injectable solutions or suspensions.

In accordance with the foregoing, the present invention also provides an agent of the invention, for use as a pharmaceutical, e.g. for the treatment of diseases induced or facilitated by CRF, such as these indicated above.

The present invention furthermore provides a pharmaceutical composition comprising an agent of the invention in association with at least one pharmaceutical carrier or diluent. Such compositions may be manufactured in conventional manner. Unit dosage forms contain, for example, from about 0.25 to about 150, preferably from 0.25 to about 25 mg of a compound according to the invention.

Moreover the present invention provides the use of an agent of the invention, for the manufacture of a medicament for the treatment of any condition mentioned herein.

In still a further aspect the present invention provides a method for the treatment of any condition mentioned above, in a subject in need of such treatment, which comprises administering to such subject a therapeutically effective amount of an agent of the invention.

The following example illustrates the invention. The temperatures are given in degrees Celsius and are uncorrected.

### EXAMPLE 1

### Cyclopropylmethyl-[7-(5,7-dimethyl-benzo[1,2,5]thiadiazol-4-yl)-2,5,6-trimethyl-7H-pyrrolo[2,3-d]pyrimidin-4-yl]-propyl-amine

A solution of 4-(4-chloro-2,5,6-trimethyl-pyrrolo[2,3-d]pyrimidin-7-yl)-5,7-dimethyl-benzo-[1,2,5]thiadiazole (1 g) and cyclopropylmethyl-propyl-amine (1.5 ml) in abs. dimethyl sulfoxide (10 ml) is stirred at 130° for 10 hours under argon. The reaction is monitored using thin layer chromatography. The reaction mixture is cooled, water (100 ml) is added and the aqueous phase is extracted with methyl t-butyl ether (2×100 ml). The organic phase is dried, evaporated and the residue is recrystallised from methanol to give the title product. Mp=136.0-136.5°.

The starting material 4-(4-chloro-2,5,6-trimethyl-pyrrolo[2,3-d]pyrimidin-7-yl)-5,7-dimethyl-benzo[1,2,5]thiadiazole is produced in 8 steps as follows:

A solution of 3,5-dimethyl-benzene-1,2-diamine (87.5 g) and N-thionylaniline (365 g) in abs. toluene (900 ml) is refluxed for 20 hours. The reaction mixture is cooled, then stirred for 10 min. with 6N hydrochloric acid (900 ml). The layers are separated and the organic layer is evaporated. The remaining oil is distilled in high vacuum to give 4,6-dimethyl-benzo[1,2,5]thiadiazole. Bp=80°, 0.1 Torr.

4,6-Dimethyl-benzo[1,2,5]thiadiazole (76.8 g) is dissolved in conc. sulphuric acid (200 ml) and cooled to 0-5°. Conc. nitric acid (25 ml, d=1.52) is added dropwise at 5-10° during 1 hour. The clear solution is poured onto ice, the resulting precipitate is filtered, washed with water, dried and recrystallised from cyclohexane to give pure 5,7-dimethyl-4-nitro-benzo[1,2,5]thiadiazole. Mp=105-106°.

A solution of 5,7-dimethyl-4-nitro-benzo[1,2,5]thiadiazole (20 g) in water (2.2 l) and ethanol (2.2 l) is heated to reflux. Sodium dithionite (180 g) is added protionwise (strongly exothermic reaction). After completion of the addition, the mixture is cooled in an ice bath and extracted with ethyl acetate. The organic layer is separated, dried and evaporated to give crude 5,7-dimethyl-benzo[1,2,5]thiadiazol-4-yl-amine that is purified by recrystallization from water. Mp=113-114°.

5,7-Dimethyl-benzo[1,2,5]thiadiazol-4-ylamine (18 g), 3-hydroxy-butan-2-one (18 g) and conc. hydrochloric acid (0.1 ml) are dissolved in cyclohexane (225 ml). The reaction mixture is heated for 4 hours using a Dean-Stark trap. The mixture is cooled for several hours, small amounts of a byproduct precipitate and are filtered off. The filtrate is evaporated, crude 3-(5,7-dimethyl-benzo[1,2,5]thiadiazole-4-ylamino)-butan-2-one is obtained as a red oil that is used for the next step without further purification.

A solution of 3-(5,7-dimethyl-benzo[1,2,5]thiadiazol-4-ylamino)-butan-2-one (24.5 g), malononitrile (7.7 g) and a catalytic amount of beta-alanine (75 mg) in abs. ethanol (150 ml) is heated for 4 hours at 80°. The reaction mixture is cooled, the resulting crystalline 2-amino-1-(5,7-dimethyl-benzo[1,2,5]thia-diazol-4-yl)-4,5-dimethyl-1H-pyrrole-3-carbonitrile is filtered off, washed with methyl t-butyl ether and recrystallised from ethyl acetate. Mp=209-211° (crude product).

2-Amino-1-(5,7-dimethyl-benzo[1,2,5]thiadiazol-4-yl)-4,5-dimethyl-1H-pyrrole-3-carbonitrile (10 g) is heated in acetic anhydride (5 ml) and glacial acetic acid (10 ml) at 50° for 2 hours. After cooling, methyl t-butyl ether (30 ml) is added. N-[3-cyano-1-(5,7-dimethyl-benzo[1,2,5]thiadiazol-4-yl)-4,5-dimethyl-1H-pyrrol-2-yl]-acetamide precipitates as colourless crystals, which are filtered, washed with methyl t-butyl ether and dried. Mp=209-211°.

N-[3-cyano-1-(5,7-dimethyl-benzo[1,2,5]thiadiazol-4-yl)-4,5-dimethyl-1H-pyrrol-2-yl]-acetamide (10 g) is heated in 85% phosphoric acid (10 ml) at 130° for 30 min. under vigorous stirring. Ice water (100 ml) is added, the resulting 7-(5,7-dimethyl-benzo[1,2,5]thiadiazol-4-yl)-2,5,6-trimethyl-3,7-dihydro-pyrrolo[2,3-d]pyrimidin-4-one is filtered off, washed with water, methanol and methyl-butyl ether and dried. Mp=357-359° (decomposition).

7-(5,7-Dimethyl-benzo[1,2,5]thiadiazol-4-yl)-2,5,6-trimethyl-3,7-dihydro-pyrrolo[2,3-d]pyrimidin-4-one (1 g) and phosphorus oxychloride (3 ml) are heated to 125° for 2.5 hours. Excess phosphorus oxychloride is distilled off in high vacuum, the residue is dissolved in ethyl acetate (20 ml), the same volume of ice water is added and the mixture is stirred for 1 hour. The layers are separated, the aqueous layer is extracted with methyl t-butyl ether (2×20 ml). The combined organic layers are dried, treated with charcoal, filtered and evaporated to give 4-(4-chloro-2,5,6-trimethyl-pyrrolo[2,3-d]pyrimidin-7-yl)-5,7-dimethyl-benzo[1,2,5]thiadiazole. Mp=169-171°.

1-8. (canceled)

9. The compound of formula A: 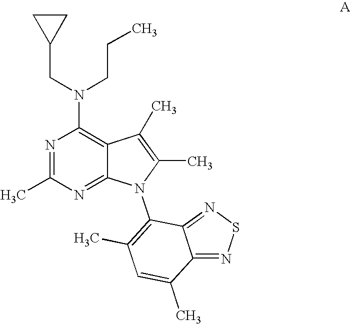
in free base or acid addition salt form.

10. A process for the preparation of the compound of formula A: 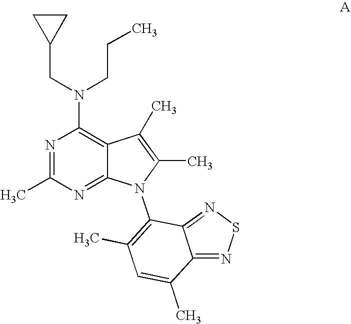
and its acid addition salts, which includes the step of reacting a compound of formula II 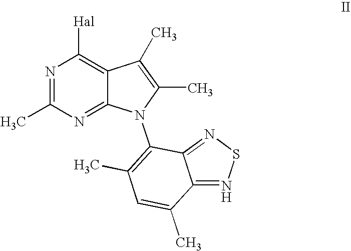
wherein Hal is halogen, with the compound of formula III 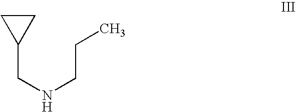
and recovering the resulting compound in free base form or acid addition salt form.

11. A pharmaceutical composition comprising the compound of claim 9 in free base or pharmaceutically acceptable acid addition salt form, in combination with a pharmaceutical carrier or diluent.
